(19) United States
(12) Patent Application Publication
(10) Pub. No.: US 2025/0319319 A1
(43) Pub. Date: Oct. 16, 2025

(54) RF ENERGY GENERATOR CAPABLE OF CONTROLLING PENETRATION DEPTH OF ENERGY

(71) Applicant: DAEYANG MEDICAL CO., LTD., Wonju-si (KR)

(72) Inventor: Jeong-Sub Yun, Wonju-si (KR)

(73) Assignee: DAEYANG MEDICAL CO., LTD., Wonju-si (KR)

(21) Appl. No.: 18/754,797

(22) Filed: Jun. 26, 2024

(30) Foreign Application Priority Data
Apr. 16, 2024 (KR) 10-2024-0050415

(51) Int. Cl. A61N 1/40; A61N 1/04

(52) U.S. Cl. CPC A61N 1/403; A61N 1/0408; A61N 1/0476

(57) ABSTRACT

Proposed is an RF energy generator capable of controlling the penetration depth of energy according to the present disclosure that includes a handpiece casing unit, a plurality of electrode units located to be spaced apart from one side of the handpiece casing unit and in contact with the skin to transmit RF energy to the skin, and an electrode gap control unit that controls the penetration depth of RF energy transmitted to the skin by adjusting the gap of the plurality of the electrode units, and that controls the penetration depth of RF energy by adjusting the gap between electrodes, enabling RF energy to penetrate the tissue in the skin over a wide range during the procedure, thereby increasing the range of the procedure and achieving more diverse procedure effects.

### CROSS REFERENCE TO RELATED APPLICATION

The present application claims priority to Korean Patent Application No. 10-2024-0050415, filed on Apr. 16, 2024, the entire contents of which are incorporated herein for all purposes by this reference.

### BACKGROUND

### Technical Field

The present disclosure relates to an RF energy generator capable of controlling a penetration depth of energy and, more specifically, to the RF energy generator capable of controlling the penetration depth of energy, and adjusting the penetration depth of RF energy by adjusting a gap between electrodes during a procedure.

### Description of the Related Art

In recent years, as the demand and interest in skincare increase, skincare devices that transmit energy into the skin to modify skin tissue conditions or improve tissue characteristics have been developed and have been gradually increasing in sales.

A variety of devices have been developed and marketed as skincare devices to treat the skin using lasers, flash lamps, ultrasound, RF energy, or the like.

A skincare device using RF energy transmits RF energy into the skin tissue and, when provided, RF current generates deep heat in the tissue within the skin while flowing along the skin tissue.

Skincare devices using RF energy raise the temperature of the skin tissue and reorganize collagen layers to improve wrinkles and enhance skin elasticity by transmitting RF energy into the skin tissue and generating deep heat in the skin tissue, and have the effect of improving the overall skin condition including providing anti-aging effects by increasing blood circulation in the skin tissue.

Skincare devices using RF energy are classified into monopolar RF energy generators having one electrode in contact with the skin and a separate ground pad, and bipolar RF energy generators having two electrodes in contact with the skin.

Since conventional bipolar RF energy generators have a structure in which the gap between a pair of RF electrodes in contact with the skin is fixed so that the transmission depth of RF energy in the skin is fixed, there is a limitation to improving the skin condition during the procedure, the procedure type is constant, and it is impossible to perform various types of procedures according to the patient's skin condition.

In addition, since conventional RF energy generators use only one handpiece when performing the procedure over a wide treatment part such as the patient's abdomen, thighs, or upper arms, there are problems of taking a long time and causing great inconvenience to the operator.

### SUMMARY

An objective of the present disclosure is to provide an RF energy generator capable of controlling a penetration depth of energy, and adjusting the penetration depth of RF energy by controlling a gap between electrodes during a procedure.

In addition, another objective of the present disclosure is to provide an RF energy generator capable of controlling a penetration depth of energy, and allowing a plurality of handpieces to be connected as needed, to be worn on a patient's body part, and to be simultaneously operated to perform a procedure.

In order to achieve the above objectives, an exemplary embodiment of an RF energy generator capable of controlling the penetration depth of energy according to the present disclosure includes a handpiece casing unit, a plurality of electrode units that are located to be spaced apart from one surface of the handpiece casing unit and that are in contact with the skin to transmit RF energy to the skin, and an electrode gap control unit that controls the penetration depth of RF energy transmitted to the skin by adjusting the gap of the plurality of electrode units in contact with the skin.

In the present disclosure, a pair of the electrode units are positioned to be tilted relative to a contact surface in contact with the skin, and the electrode gap control unit includes an electrode rotation unit that rotates the tilted electrode unit, wherein the electrode rotation unit rotates the tilted electrode unit to adjust the gap at one end side of the electrode units in contact with the skin.

In the present disclosure, the pair of the electrode units are positioned to be tilted while facing each other, but are positioned to be tilted in opposite directions.

In this disclosure, the electrode rotation unit includes a rotation motor unit mounted within the handpiece casing unit, a center gear unit rotated by receiving a rotation force of the rotation motor unit, and a plurality of electrode rotation gear units being mounted on an electrode rotation shaft unit located on the other end side of the electrode unit and being interlocked with the center gear unit.

In the present disclosure, the pair of the electrode units are capable of being rotated at different rotation speeds.

In the present disclosure, the plurality of the electrode units have a plurality of electrode pairs, and at least one electrode pair among the plurality of the electrode pairs has a direction misaligned with the other electrode pairs, such that the plurality of the electrode pairs can form gaps different from each other when rotated.

The present disclosure further includes a wearing band unit for wearing the handpiece casing unit on a patient's body, and a casing connection unit for detachably coupling the wearing band unit to the handpiece casing unit.

In the present disclosure, the wearing band unit is detachably coupled to the handpiece casing unit by the casing connection unit, and is rotatably hinged thereto.

In the present disclosure, the casing connection unit includes a first hinge body unit that is positioned on one side of either of the handpiece casing units and the wearing band unit and that is provided with a protruding hinge shaft, and a second hinge body unit that is positioned on the other side of either of the handpiece casing unit and the wearing band unit and that is provided with a shaft insertion unit where the hinge shaft is inserted.

In the present disclosure, the first hinge body unit and the second hinge body unit are respectively provided on both sides of the handpiece casing unit, and the second hinge body unit is provided on the other side of the handpiece casing unit, so that the plurality of the handpiece casing units allows the first hinge body unit to be connected to the second hinge body unit to be connected to each other.

The present disclosure is capable of controlling the penetration depth of RF energy by adjusting the gap between electrodes during the procedure, enabling RF energy to penetrate the tissue in the skin over a wide range during the procedure, thereby increasing the range of the procedure and achieving more diverse procedure effects.

In addition, the present disclosure enables a plurality of handpieces to be connected as needed, to be worn on a patient's body part, and to simultaneously be operated to perform a procedure, thereby achieving procedural convenience and procedural efficiency.

### BRIEF DESCRIPTION OF THE DRAWINGS

FIG. 1 is a perspective view showing an exemplary embodiment of an RF energy generator capable of controlling a penetration depth of energy according to the present disclosure.

FIG. 2 is an exploded perspective view showing an exemplary embodiment of an RF energy generator capable of controlling a penetration depth of energy according to the present disclosure.

FIGS. 3 to 5B are schematic diagrams showing different examples in which two electrode units are adjusted for a gap by an operation of an electrode gap control unit in an RF energy generator capable of controlling a penetration depth of energy according to the present disclosure.

FIG. 6 is a perspective view showing another exemplary embodiment of an RF energy generator capable of controlling a penetration depth of energy according to the present disclosure.

### DETAILED DESCRIPTION OF THE DISCLOSURE

Hereinafter, preferred exemplary embodiments of the present disclosure will be described in detail with reference to the accompanying drawings. However, the technical idea of the present disclosure is not limited to the exemplary embodiments described herein, and may be embodied in other forms. Rather, the exemplary embodiments introduced herein are provided so that the disclosed content may be thorough and complete, and so that the spirit of the present disclosure may be sufficiently conveyed to those skilled in the art.

In the present specification, when it is mentioned that a component is referred to as being on another component, it means that it may be formed directly on the other component or that a third component may be interposed between them. In addition, the thickness of the shapes and regions in the drawings is exaggerated for an effective description of the technical content.

In addition, terms such as a first, a second, and a third are used to describe various components in various exemplary embodiments of the present specification, but these components should not be limited by these terms. These terms are merely used to distinguish one component from another. Thus, what is referred to as a first component in one exemplary embodiment may be referred to as a second component in another exemplary embodiment. Each exemplary embodiment described and illustrated herein also includes a complementary exemplary embodiment thereof. In addition, “and/or” in the present specification is used having a meaning including at least one of the components listed before and after.

Singular expressions in the specification include plural expressions unless the context clearly dictates otherwise. In addition, the term “include” or “have” is intended to specify the existence of a feature, number, step, component, or combination thereof described in the specification, and should not be construed to exclude the possibility of the presence or addition of one or more other features, numbers, steps, components, or combinations thereof. In addition, the term “connection” in the present specification is used to include both indirect and direct connections of a plurality of components.

In addition, the detailed description will be omitted when it is determined that a detailed description of a related known function or configuration in describing the present disclosure may unnecessarily obscure the gist of the present disclosure.

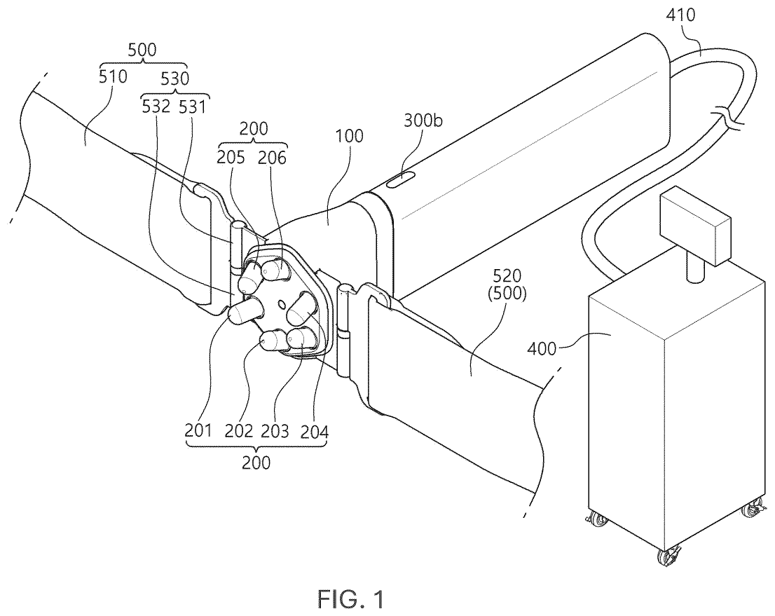

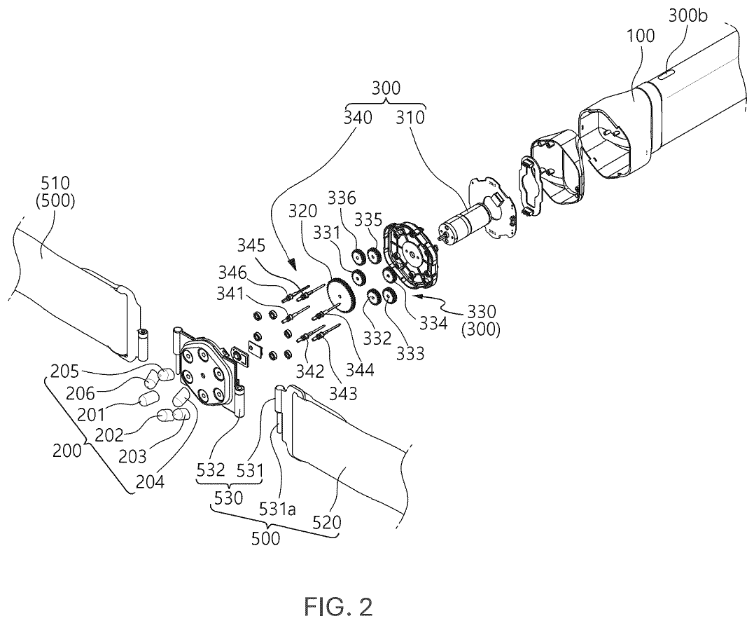

FIG. 1 is a perspective view showing an exemplary embodiment of an RF energy generator capable of controlling a penetration depth of energy according to the present disclosure, and FIG. 2 is an exploded perspective view showing an exemplary embodiment of an RF energy generator capable of controlling a penetration depth of energy according to the present disclosure.

An exemplary embodiment of an RF energy generator capable of controlling a penetration depth of energy according to the present disclosure will be described in detail below with reference to FIGS. 1 and 2.

An exemplary embodiment of an RF energy generator capable of controlling a penetration depth of energy according to the present disclosure may include a handpiece casing unit 100, wherein the handpiece casing unit 100 has a shape that may be held and used by an operator.

It should be noted that the handpiece casing unit 100 may be implemented by various modifications to a known structure in a known skincare device using an RF energy generator, so a more detailed explanation will be omitted.

An electrode mount surface where a plurality of electrode units 200 in contact with the skin are located may be located on one end side of the handpiece casing unit 100 and a control cable body 410 that is connected to a control main body 400 may be connected to the other end side of the handpiece casing unit 100, wherein the control main body applies electric power to the electrode unit 200 and controls the operations of the electrode unit 200.

That is, an exemplary embodiment of an RF energy generator capable of controlling a penetration depth of energy according to the present disclosure may further include a control main body 400, which is connected to the handpiece casing unit 100 by the control cable body 410 and controls the operations of the plurality of the electrode units 200 and the electrode gap control unit 300.

The control main body unit 400 may include an RF signal generator that generates RF energy by applying electrical power to the plurality of the electrode units 200, and may be implemented by various modifications to a known structure in a known skincare device using an RF energy generator, so a more detailed explanation will be omitted.

In addition, the inside of the handpiece casing unit 100 may be provided with a control board (not shown) that is electrically connected to the plurality of the electrode units 200 and the control main body unit 400, respectively, and the control main body unit 400 may control the operations of the plurality of the electrode units 200 and the electrode gap control unit 300, including the control board located inside the handpiece casing unit 100.

As an example, the plurality of the electrode units 200 may include at least two pairs of electrodes to which a (+) power source and a (−) power source are respectively connected. An exemplary embodiment of an RF energy generator capable of controlling a penetration depth of energy according to the present disclosure may include three pairs of electrodes to which the (+) power source and the (−) power source are respectively connected and, more specifically, may include one pair of the electrode units 200 including a first electrode unit 201 and a second electrode unit 202 to which the (+) power source and the (−) power source are respectively connected, another pair of the electrode units 200 including a third electrode unit 203 and a fourth electrode unit 204 to which the (+) power source and the (−) power source are respectively connected, and the other pair of the electrode units 200 including a fifth electrode unit 205 and a sixth electrode unit 206 to which the (+) power source and the (−) power source are respectively connected.

That is, as an example, the plurality of the electrode units 200 may include a total of six electrodes, and two electrode units 200 may be paired together to totally include three electrode pairs, as an example.

An exemplary embodiment of an RF energy generator capable of controlling a penetration depth of energy according to the present disclosure may include the electrode gap control unit 300 that controls the penetration depth of RF energy transmitted to the skin by adjusting the gap of the plurality of the electrode units 200 in contact with the skin.

As an example, the electrode gap control unit 300 may control the penetration depth of RF energy transmitted to the skin between a pair of the electrode units 200 in contact with the skin by adjusting the distance between the pair of the electrode units 200 to which the (+) power source and the (−) power source are respectively connected while being in contact with the skin.

When the gap between one pair of electrodes is large, the penetration depth of RF energy transmitted into the skin's tissue becomes deeper, and when the gap between one pair of electrodes becomes narrow, the penetration depth of RF energy transmitted into the skin's tissue becomes shallow.

That is, an exemplary embodiment of an RF energy generator capable of controlling a penetration depth of energy according to the present disclosure may be a bipolar RF energy generator that includes at least one pair of the electrode units 200 to which the (+) power source and the (−) power source are respectively connected while being in contact with the skin, and that transmits RF energy generated between one pair of the electrode units 200 to the skin, and may control the penetration depth of RF energy transmitted to the skin by adjusting the distance between one pair of the electrode units 200 in contact with the skin with the electrode gap control unit 300.

More specifically, one pair of the electrode units 200 may be positioned to be tilted relative to the contact surface in contact with the skin, and the electrode gap control unit 300 may include an electrode rotation unit 300a that rotates the tilted electrode unit 200 and may adjust the gap at one end side of the electrode unit 200 in contact with the skin by rotating the tilted electrode unit with the electrode rotation unit 300a.

The plurality of the electrode units 200 may be positioned to protrude relative to an electrode mount surface of a plane located at one end side of the handpiece casing unit 100, but may be positioned to be tilted at a preset angle.

Also, the electrode rotation unit 300a may allow one end side of the electrode unit 200 to move in a circle with a certain radius by rotating the electrode unit 200 centering the other end side of the electrode unit 200.

The electrode gap control unit 300 may further include a rotation control switch unit 300b that is provided in the handpiece casing unit 100 and that is operated by the operator to control the operations of the electrode rotation unit 300a.

The rotation control switch unit 300b may turn on and off the operations of the electrode rotation unit 300a by allowing the operator to manipulate with the fingers in a state of holding the handpiece casing unit 100 in the hand.

One pair of the electrode units 200 may be positioned to face each other and to be tilted, but, to be tilted in opposite directions, so that the distance adjustment range can be set to the maximum.

When one pair of the electrode units 200 are rotated by the electrode rotation unit 300a and positioned to face each other inside so that one end faces the other, the minimum distance may be set and, when one end is positioned to face each other outside so that one end faces the other, the maximum distance may be set.

That is, one pair of the electrode units 200 to which the (+) power source and the (−) power source are respectively connected may be positioned to be at a minimum distance when one end side in contact with the skin is tilted inward while facing each other, or may be tilted in completely opposite directions to be positioned at a maximum distance by being tilted outward while facing each other.

One pair of the electrode units 200 may be rotated by the electrode rotation unit 300 with one end side located to be tilted and protrude from one end side of the handpiece casing unit 100 being in contact with the skin, so that the electrode gap may be adjusted.

In addition, the electrode rotation unit 300a may include a rotation motor unit 310 mounted within the handpiece casing unit 100, a center gear unit 320 rotated by receiving the rotation force of the rotation motor unit 310, and a plurality of electrode rotation gear units 330 being mounted on an electrode rotation shaft unit 340 located on the other end side of the electrode units 200 and being interlocked with the center gear unit 320.

The electrode rotation gear unit 330 may be mounted on the electrode rotation shaft unit 340 located on the other end side of the electrode units 200, and may rotate centering on the electrode rotation shaft unit 340 by receiving the rotation force of the rotation motor unit 310 from the center gear unit 320 while interlocking with the center gear unit 320.

The plurality of the electrode units 200 may have the plurality of the electrode rotation gear units 330 mounted on each electrode rotation shaft unit 340, and the plurality of the electrode rotation gear units 330 may be disposed to be spaced apart from the outer circumference of the center gear unit 320 in the circumferential direction to interlock with the center gear unit 320.

The plurality of the electrode units 200 may be together rotated centering on the electrode rotation shaft unit 340 by the rotation of the plurality of the electrode rotation gear units 330 interlocked with the center gear unit 320.

As an example, when provided with a total of six electrode units 200, including a pair of the electrode units 200 including a first electrode unit 201 and a second electrode unit 202 to which the (+) power source and the (−) power source are respectively connected, a pair of the electrode units 200 including a third electrode unit 203 and a fourth electrode unit 204 to which the (+) power source and the (−) power source are respectively connected, and a pair of the electrode units 200 including a fifth electrode unit 205 and a sixth electrode unit 206 to which the (+) power source and the (−) power source are respectively connected, the plurality of the electrode rotation gear units 330 may include a first electrode rotation gear unit 331 mounted on a first electrode rotation shaft unit 341 of a first electrode unit 201, a second electrode rotation gear unit 332 mounted on a second electrode rotation shaft unit 342 of a second electrode unit 202, a third electrode rotation gear unit 333 mounted on a third electrode rotation shaft unit 343 of a third electrode unit 203, a fourth electrode rotation gear unit 334 mounted on a fourth electrode rotation shaft unit 344 of a fourth electrode unit 204, a fifth electrode rotation gear unit 335 mounted on a fifth electrode rotation shaft unit 345 of a fifth electrode unit 205, and a sixth electrode rotation gear unit 336 mounted on a sixth electrode rotation shaft unit 346 of a sixth electrode unit 206.

That is, a pair of the first electrode unit 201 and the second electrode unit 202 tilted facing each other may be respectively rotated by receiving the rotation force of the center gear unit 320 via the first electrode rotation gear unit 331 and the second electrode rotation gear unit 332, so that the penetration depth of RF energy transmitted into the skin may change as the gap on one end side in contact with the skin changes.

In addition, a pair of the third electrode unit 203 and the fourth electrode unit 204 tilted facing each other may be respectively rotated by receiving the rotation force of the center gear unit 320 via the third electrode rotation gear unit 333 and the fourth electrode rotation gear unit 334, so that the penetration depth of RF energy transmitted into the skin may change as the gap on one end side in contact with the skin changes.

In addition, a pair of the fifth electrode unit 205 and the sixth electrode unit 206 tilted facing each other may be respectively rotated by receiving the rotation force of the center gear unit 320 via the fifth electrode rotation gear unit 335 and the sixth electrode rotation gear unit 336, so that the penetration depth of RF energy transmitted into the skin may change as the gap on one end side in contact with the skin changes.

One pair of the electrode units 200 may be rotated at different rotation speeds, such that the gap of one end side in contact with the skin changes to more various ranges and the penetration depth of RF energy penetrated into the skin may be adjusted to more various ranges.

One pair of the electrode rotation gear units 330 connected to one pair of the electrode units 200 may have different diameters or different tooth ratios, such that one pair of the electrode units 200 can be rotated at different rotation speeds.

In addition, as an example, at least one electrode pair among the plurality of the electrode pairs may have a direction that is misaligned with the other electrode pairs, so that the plurality of the electrode pairs can form different gaps when rotated.

The plurality of the electrode pairs may be tilted and positioned to have a misaligned direction while being rotated simultaneously by the rotation motor unit 310, so that each electrode pair is rotated while the gap of one end side in contact with the skin has a gap different from each other when rotated.

The plurality of the electrode pairs may be positioned in a misaligned relationship, and may enable more diverse and wide-ranging treatments by transmitting RF energy at different depths to the treatment part when rotated simultaneously by the operations of the rotation motor unit 310.

In addition, the plurality of the electrode pairs may generate the effect of massaging the skin when rotating with a preset radius while being tilted in contact with the skin.

That is, the plurality of the electrode pairs 200 may be positioned to protrude from one end side of the handpiece casing unit 100 and transmit RF energy to the skin while being in contact with the skin and, in order to be in close contact with the skin, may be in contact with the skin in a state that the skin is pressurized at a certain pressure by the operator or a wearing band unit that will be described later.

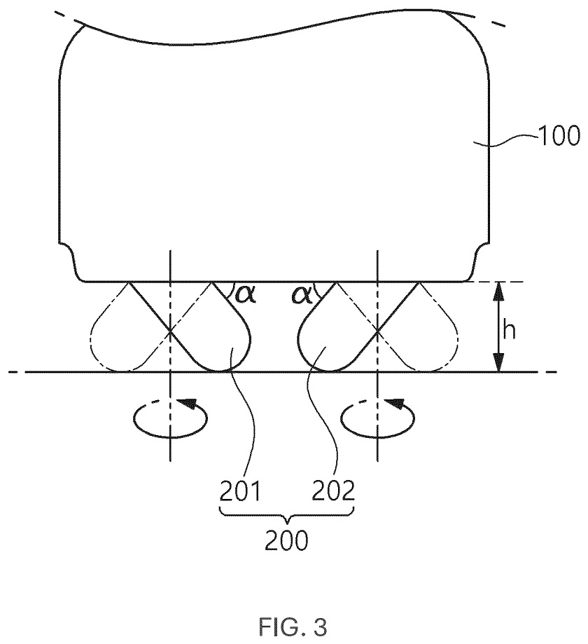

Also, since being in contact with the skin in a pressurized state by the force of the operator or the wearing pressure of the wearing band unit to be described later and being rotated having a preset radius in a state while being tilted in contact with the skin, the plurality of the electrode units 200 may increase the effectiveness of the treatment by generating a massage effect at the same time when transmitting RF energy to the skin and may greatly improve the satisfaction of the treatment by reducing pain during RF energy procedure. FIGS. 3 to 5B are schematic diagrams showing different examples in which a pair of electrode units 200, that is, a first electrode unit 201 and a second electrode unit 202, are adjusted for a gap by an operation of an electrode gap control unit 300 in an RF energy generator capable of controlling a penetration depth of energy according to the present disclosure.

Referring to FIG. 3, an example is shown in which a pair of electrode units 200 are each tilted at the same angle and protrude at the same height on the electrode mount surface of the handpiece casing unit 100.

The first electrode unit 201 and the second electrode unit 202 may be positioned to be tilted and face each other on the electrode mount surface of the plane, and each may rotate centering on the electrode rotation shaft unit on the other end side, thereby making the gap on one end side in contact with the skin vary and adjusting the penetration depth of RF energy penetrating into the skin.

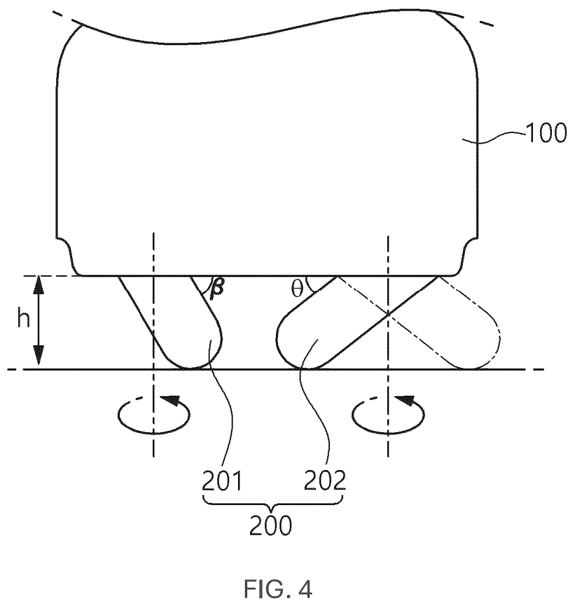

In addition, referring to FIG. 4, one pair of the electrode units 200, that is, the first electrode unit 201 and the second electrode unit 202 may be positioned to be tilted at different angles to further widen the difference between the minimum gap and the maximum gap, thereby forming a wider range of penetration depths of RF energy into the skin.

The first electrode unit 201 among the pair of the electrode units 200 may be located to be tilted at a first angle relative to the electrode mount surface of the plane, and the second electrode unit 202 among the pair of the electrode units 200 may be located to be tilted at a second angle lower than the first angle relative to the electrode mount surface of the plane, such that one end side of each electrode unit 200 in contact with the skin may be rotated having a radius different from each other and the minimum gap and maximum gap become wider relative to a structure with the same angle since each electrode unit 200 is individually rotated.

Also, the first electrode unit 201 and the second electrode unit 202 may be tilted at different angles, but may be positioned on the electrode mount surface at the same height, thereby being in stable contact with the skin.

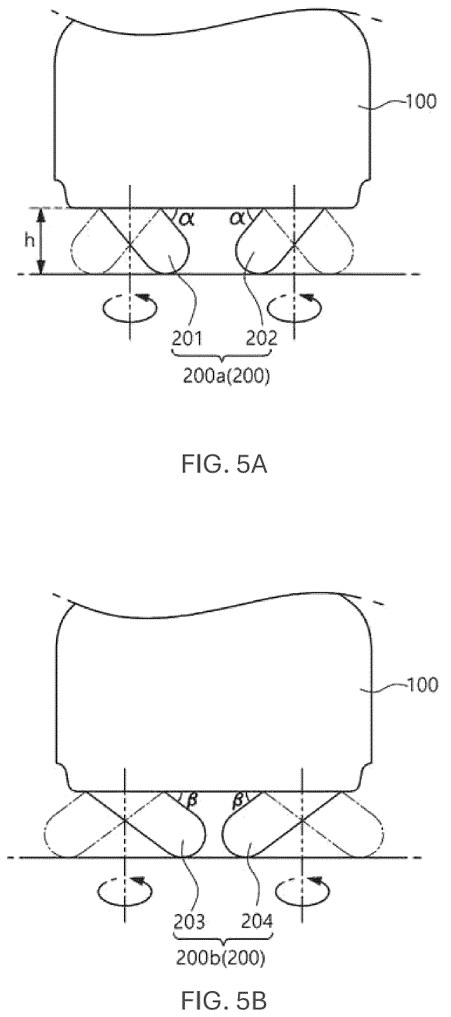

Meanwhile, as an example, referring to FIGS. 5A and 5B, when the plurality of the electrode units 200 are composed of a plurality of pairs, the range of the gap change during the rotation of at least one pair of the electrode units 200 may be different from the range of the gap change during the rotation of another pair of the electrode units 200.

FIGS. 5A and 5B are views showing a first electrode pair 200a and a second electrode pair 200b positioned to protrude from an electrode mount surface and, as an example, the first electrode pair 200a includes a pair of electrode units 200 being tilted at a first angle, that is, the first electrode unit 201 and the second electrode unit 202, and the second electrode pair 200b includes a pair of electrode units 200 being tilted at a second angle different from the first angle, that is, the third electrode unit 203 and the fourth electrode unit 204.

Also, although not shown, one electrode of the first electrode pair 200a may be tilted at a first angle, the other electrode may be tilted at a third angle different from the first angle, one electrode of the second electrode pair 200b may be tilted at a second angle, and the other electrode may be tilted at a fourth angle.

The third angle and the fourth angle may be the same angle or may be different angles.

The pair of the electrode units 200 of the first electrode pair 200a and the pair of electrode units 200 of the second electrode pair 200b may be tilted at different angles, so that when each electrode unit 200 is rotated, the rotation radius of the electrode units 200 of the first electrode pair, that is, the first electrode unit 201 and the second electrode unit 202 is different from the rotation radius of the electrode units 200 of the second electrode pair, that is, the third electrode unit 203 and the fourth electrode unit 204, thereby having the range of the gap change different from each other when rotated.

The plurality of the electrode pairs 200a, 200b may be all tilted at different angles and may have all the range of the gap change different from each other when rotated.

In addition to adjusting the angle of the electrode unit 200, it should be noted that other modifications are possible in which the plurality of the electrode pairs 200a, 200b may have the range of the gap change different from each other. The RF energy generator capable of controlling a penetration depth of energy according to the present disclosure may enable more diverse procedures by individually and differently adjusting the range of penetration into the skin since at least two pairs of the electrode units 200 are tilted at different angles so that the gap between the electrodes is adjusted to the range of the gap change different from each other when rotated.

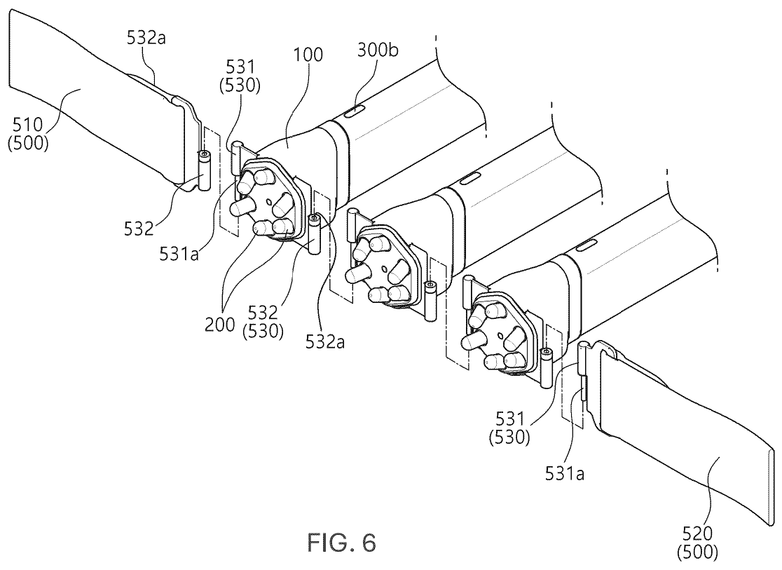

FIG. 6 is a perspective view showing another exemplary embodiment of an RF energy generator capable of controlling a penetration depth of energy according to the present disclosure and, referring to FIGS. 1, 2, and 6, an exemplary embodiment of a RF energy generator capable of controlling a penetration depth of energy according to the present disclosure may further include a wearing band unit 500 for wearing the handpiece casing unit 100 on a patient's body, and a casing connection unit 530 detachably coupling the wearing band unit 500 to the handpiece casing unit 100.

The wearing band unit 500 may include a first band member 510 detachably connected to one side of the handpiece casing unit 100, a second band member 520 detachably connected to the other side of the handpiece casing unit 100, and a band connection unit (not shown) connecting the first band member 510 and the second band member 520, wherein both end sides after wrapping a body part are connected to each other to be worn on the patient's body and to allow the position of the handpiece casing unit 100 to be fixed at the patient's treatment part.

Although not shown, the band connection unit may be, as an example, a Velcro tape and may include a male Velcro tape positioned on one side of either of the first band member 510 and the second band member 520 and a female Velcro tape positioned on the other side of either of the first band member 510 and the second band member 520.

The male Velcro tape and the female Velcro tape may be attached to each other to connect both end sides of the wearing band unit 500, so that the wearing band unit 500 is worn on the patient in a state of wrapping the patient's treatment part and the position of the handpiece casing unit 100 is fixed at the patient's treatment part.

The first band member 510 and the second band member 520 may be adjustable in length to be worn around a body part by adjusting the attachment area of the male Velcro tape and female Velcro tape, thereby being wearable on various body parts such as arms, legs, waist, and chest.

Although not shown, the band connection unit may be implemented by various modifications to known band connectors, such as a clamp structure or a button structure that connects the bands to wrap a body part and wears the band on the body, so a more detailed description is omitted.

As an example, the casing connection unit 530 may be rotatably connected by rotatably hinging the wearing band unit 500 to the handpiece casing unit 100.

The wearing band unit 500 may be detachably coupled to the handpiece casing unit 100 by a casing connection unit 530, but may be rotatably hinged, so that the handpiece casing unit 100 can be easily worn on the patient's body part and the feeling of wearing is comfortable after the handpiece casing unit 100 is worn on the patient's body part.

The casing connection unit 530 may include a first hinge body unit 531 being located on one side of either of the handpiece casing unit 100 and the wearing band unit 500 and being provided with a protruding hinge shaft 531a, and a second hinge body unit 532 being located on one side of either of the handpiece casing unit 100 and the wearing band unit 500 and being provided with a shaft insertion unit 532a into which the hinge shaft 531a is inserted.

Each of the first hinge body unit 531 and the second hinge body unit 532 may have a circular rod shape and may be arranged in a straight line when combined, the hinge shaft 531a may stand vertically and protrude from the center of the lower surface of the first hinge body unit 531, and the shaft insertion unit 532a may be positioned to be open on the upper surface of the second hinge body unit 532.

The first hinge body unit 531 may be moved downward from the upper side of the second hinge body unit 532, so that the hinge shaft 531a is inserted into the shaft insertion unit 532a to be coupled, and may be moved in the opposite direction to be simply separated from the second hinge body unit 532.

The casing connection unit 530 may be respectively located on both sides of the handpiece casing unit 100 to connect the first band member 510 and the second band member 520 respectively.

That is, the first hinge body unit 531 may be provided on one side of the handpiece casing unit 100, and the second hinge body unit 532 may be provided on the first band member 510, so that the first band member 510 is rotatably hinged to one side of the handpiece casing unit 100.

In addition, the second hinge body unit 532 may be provided on the other side of the handpiece casing unit 100, and the first hinge body unit 531 may be provided on the second band member 520, so that the second band member 520 is rotatably hinged to the other side of the handpiece casing unit 100.

The first band member 510 may be simply connected to one side of the handpiece casing unit 100 when the first hinge body unit 531 is coupled to the second hinge body unit 532 on one end side, and the second band member 520 may be connected to one side of the handpiece casing unit 100 when the first hinge body unit 531 is coupled to the second hinge body unit 532 on one end side.

In addition, the first hinge body unit 531 and the second hinge body unit 532 may be respectively provided on both sides of the handpiece casing unit 100, so that a plurality of handpiece casing units 100 can be easily coupled and connected to each other.

The handpiece casing units 100 may be connected to each other by allowing the first hinge body unit 531 on one end side to be coupled to the second hinge body unit 532 of another handpiece casing unit 100, or may be connected to each other by allowing the second hinge body unit 532 on the other end to be coupled to the first hinge body unit 531 of another handpiece casing unit 100.

That is, an exemplary embodiment of a RF energy generator capable of controlling a penetration depth of energy according to the present disclosure may selectively connect a plurality of the handpiece casing units 100 depending on the treatment area of the treatment part, and may be used by connecting the wearing band units to the handpiece casing units 100 located at both ends of the plurality of the handpiece casing units 100 connected to each other.

An exemplary embodiment of a RF energy generator capable of controlling a penetration depth of energy according to the present disclosure may enable a large treatment area to be simultaneously treated by connecting the plurality of the handpiece casing units 100 according to the treatment area of the treatment part, thereby improving treatment convenience and greatly reducing treatment time.

In addition, an exemplary embodiment of a RF energy generator capable of controlling a penetration depth of energy according to the present disclosure may improve the convenience and stability of the procedure by fixing the handpiece casing unit 100 to the body with the wearing band unit and by completely and closely contacting the treatment part.

The present disclosure may be capable of controlling the penetration depth of RF energy by adjusting the gap between electrodes during the procedure, enabling RF energy to penetrate the tissue in the skin over a wide range during the procedure, thereby increasing the range of the procedure and achieving more diverse procedure effects.

In addition, the present disclosure may enable a plurality of handpieces to be connected as needed, to be worn on a patient's body part, and to be operated simultaneously to perform a procedure, thereby achieving procedural convenience and procedural efficiency.

Although the present disclosure has been described above in detail using preferred exemplary embodiments, the scope of the present disclosure is not limited to a specific exemplary embodiment and should be interpreted according to the accompanying claims. In n addition, those who have acquired ordinary knowledge in the art should understand that many modifications and variations are possible without departing from the scope of the present disclosure.

What is claimed is:

1. An RF energy generator capable of controlling a penetration depth of energy, the RF energy generator comprising:
a handpiece casing unit;
a plurality of electrode units being located to be spaced apart from one surface of the handpiece casing unit and transmitting RF energy to skin while being in contact with the skin; and
an electrode gap control unit controlling the penetration depth of RF energy transmitted to the skin by adjusting a gap of the plurality of the electrode units in contact with the skin.

2. The RF energy generator of claim 1, wherein a pair of the electrode units are positioned to be tilted relative to a contact surface in contact with the skin, and
the electrode gap control unit comprises an electrode rotation unit rotating the tilted electrode units, wherein the electrode rotation unit rotates the tilted electrode units to adjust the gap at one end side of the electrode unit in contact with the skin.

3. The RF energy generator of claim 2, wherein the pair of the electrode units are positioned to be tilted while facing each other, but are positioned to be tilted in opposite directions.

4. The RF energy generator of claim 2, wherein the electrode rotation unit comprises:
a rotation motor unit mounted within the handpiece casing unit;
a center gear unit rotated by receiving a rotation force of the rotation motor unit; and
a plurality of electrode rotation gear units being mounted on an electrode rotation shaft unit located on the other end side of the electrode units and being interlocked with the center gear unit.

5. The RF energy generator of claim 2, wherein the pair of the electrode units are capable of being rotated at different rotation speeds.

6. The RF energy generator of claim 2, wherein the plurality of the electrode units have a plurality of electrode pairs,
wherein at least one electrode pair among the plurality of the electrode pairs has a direction misaligned with the other electrode pairs, such that the plurality of the electrode pairs can form the gap different from each other when rotated.

7. The RF energy generator of claim 1, further comprising:
a wearing band unit for wearing the handpiece casing unit on a patient's body; and
a casing connection unit for detachably coupling the wearing band unit to the handpiece casing unit.

8. The RF energy generator of claim 7, wherein the wearing band unit is detachably coupled to the handpiece casing unit by the casing connection unit, and is rotatably hinged thereto.

9. The RF energy generator of claim 7, wherein the casing connection unit comprises:
a first hinge body unit being positioned on one side of either of the handpiece casing unit and the wearing band unit and being provided with a protruding hinge shaft; and
a second hinge body unit being positioned on the other side of either of the handpiece casing unit and the wearing band unit and being provided with a shaft insertion unit where the hinge shaft is inserted.

10. The RF energy generator of claim 9, wherein the first hinge body unit and the second hinge body unit are respectively provided on both sides of the handpiece casing unit, and the second hinge body unit is provided on the other side of the handpiece casing unit, so that the plurality of the handpiece casing units enable the first hinge body unit to be coupled to the second hinge body unit to be connected to each other.
